(19) United States
(12) Patent Application Publication
(10) Pub. No.: US 2021/0091451 A1
(43) Pub. Date: Mar. 25, 2021

(54) RECEIVING ANTENNA SYSTEM AND CAPSULE ENDOSCOPE SYSTEM

(71) Applicant: OLYMPUS CORPORATION, Tokyo (JP)

(72) Inventors: Naoya HIGAKI, Tokyo (JP); Toru MIYAZONO, Tokyo (JP)

(73) Assignee: OLYMPUS CORPORATION, Tokyo (JP)

(21) Appl. No.: 16/951,131

(22) Filed: Nov. 18, 2020

Related U.S. Application Data
(63) Continuation of application No. PCT/JP2019/007685, filed on Feb. 27, 2019.

(30) Foreign Application Priority Data
May 23, 2018 (JP) 2018-099135

(51) Int. Cl. H01Q 1/22; H01Q 1/32; H01Q 1/40; H01Q 1/38

(52) U.S. Cl. CPC H01Q 1/22; H01Q 1/38; H01Q 1/40; H01Q 1/32

(57) ABSTRACT

A receiving antenna system includes: a receiving antenna including: an antenna element configured to receive a radio signal transmitted from a medical device introduced into a subject; and a base material on which the antenna element is mounted, the base material including a first base material, and a second base material; and a holder attached to the subject and configured to house the receiving antenna, wherein the antenna element is mounted on the first base material or the second base material, one of the first base material and the second base material has higher effective permittivity than effective permittivity of another one of the first base material and the second base material, and the first base material, the antenna element and the second base material are arranged in this order from a subject side.

This application is a continuation of PCT international application No. PCT/JP2019/007685 filed on Feb. 27, 2019, which designates the United States, incorporated herein by reference, and which claims the benefit of priority from Japanese Patent Application No. 2018-099135, filed on May 23, 2018, incorporated herein by reference.

### BACKGROUND

The present disclosure relates to a receiving antenna system and a capsule endoscope system.

In the field of endoscopes, a capsule endoscope configured such that an imaging function, a radio communication function, and the like are incorporated in a capsule-shaped casing with a size that is introducible into a digestive tract of a subject, such as a patient, has been known. The capsule endoscope is swallowed from a mouth of the subject, moves inside the subject, such as a digestive tract, by peristaltic movement or the like, sequentially captures images of the inside of the subject, generate pieces of image data, and sequentially transmits the pieces of image data by radio. The pieces of image data that are transmitted by radio by the capsule endoscope are received by a receiving device via a plurality of receiving antennas that are arranged on an exterior portion of the subject. Each of the receiving antennas is held by an antenna holder and fixed to a body surface of the subject.

Here, as a technique of suppressing transmission loss due to variation in characteristic impedance in radio communication, a technique of increasing electric power to be distributed to other transmission antennas that are not affected by a subject and reducing variation in electrical characteristics of the entire receiving antennas has been known (for example, see Japanese Patent No. 4538651).

### SUMMARY

According to one aspect of the present disclosure, there is provided a receiving antenna system including: a receiving antenna including: an antenna element configured to receive a radio signal transmitted from a medical device introduced into a subject; and a base material on which the antenna element is mounted, the base material including a first base material, and a second base material; and a holder attached to the subject and configured to house the receiving antenna, wherein the antenna element is mounted on the first base material or the second base material, one of the first base material and the second base material has higher effective permittivity than effective permittivity of another one of the first base material and the second base material, and the first base material, the antenna element and the second base material are arranged in this order from a subject side.

The above and other features, advantages and technical and industrial significance of this disclosure will be better understood by reading the following detailed description of presently preferred embodiments of the disclosure, when considered in connection with the accompanying drawings.

### BRIEF DESCRIPTION OF THE DRAWINGS

FIG. 1 is a schematic diagram illustrating a schematic configuration of a capsule endoscope system including an antenna holder according to an embodiment;

FIG. 2 is a schematic diagram illustrating an overall configuration of the antenna holder illustrated in FIG. 1;

FIG. 3 is a schematic diagram illustrating an overall configuration of receiving antennas illustrated in FIG. 1;

FIG. 4 is an enlarged view of an antenna unit illustrated in FIG. 2;

FIG. 5 is a partial cross-sectional view taken along a line A-A in FIG. 4;

FIG. 6 is a diagram for explaining a specimen for analyzing a difference in impedance variation;

FIG. 7 is a graph for explaining a difference in impedance variation between with and without a dielectric body;

FIG. 8 is a diagram schematically illustrating a configuration of a main part of an antenna holder that holds receiving antennas according to a first modification of the embodiment;

FIG. 9 is a partial cross-sectional view schematically illustrating a configuration of a main part of an antenna holder that holds receiving antennas according to a second modification of the embodiment;

FIG. 10 is a diagram schematically illustrating a configuration of a main part of an antenna holder that holds receiving antennas according to a third modification of the embodiment;

FIG. 11 is a diagram schematically illustrating a configuration of a main part of an antenna holder that holds receiving antennas according to a fourth modification of the embodiment; and

FIG. 12 is a diagram schematically illustrating a configuration of a main part of an antenna holder that holds receiving antennas according to a fifth modification of the embodiment;

### DETAILED DESCRIPTION

A capsule endoscope system using a capsule endoscope as a medical device will be described below as an embodiment. In description of the drawings, the same components are denoted by the same reference symbols. In addition, the drawings are schematic, and a relation between a thickness and a width of each of the components, ratios among the components, and the like may be different from actual ones.

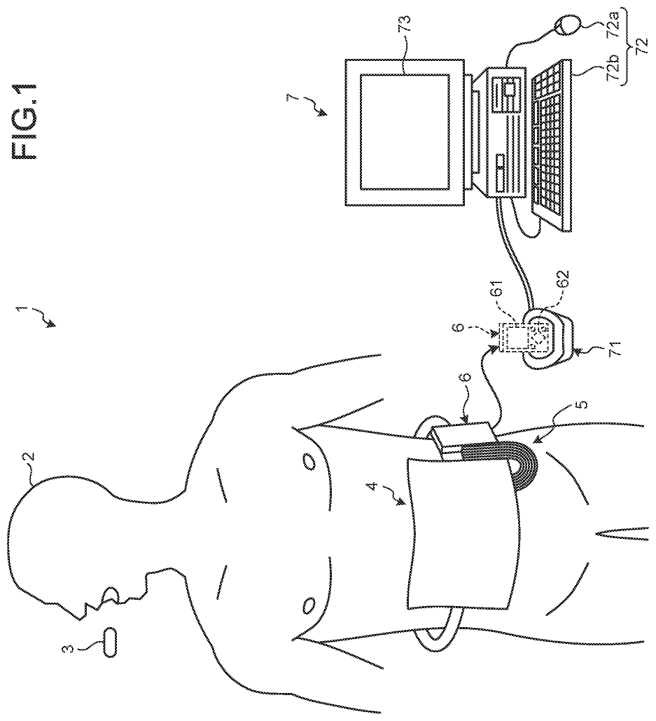

FIG. 1 is a schematic diagram illustrating an overall configuration of a capsule endoscope system including an antenna holder according to the embodiment. A capsule endoscope system 1 illustrated in FIG. 1 includes a capsule endoscope 3 as a medical device that is introduced into a subject 2, an antenna holder 4 that is attached to the subject 2 and holds a plurality of receiving antennas, an antenna device 5 that is mounted on the antenna holder 4 and includes the plurality of receiving antennas each receiving a radio signal that is transmitted from the capsule endoscope 3 introduced into the subject 2, a receiving device 6 to which the antenna device 5 is connected in an attachable/detachable manner, which performs a predetermined process on the radio signal received by the antenna device 5, and which records or displays the radio signal, and an image processing apparatus 7 that performs a process corresponding to image data of the inside of the subject 2 captured by the capsule endoscope 3 and/or performs displaying. The antenna holder 4 and the receiving antennas included in the antenna device 5 constitute an antenna unit.

The capsule endoscope 3 has an imaging function to capture images of the inside of the subject 2, and a radio function to transmit a radio signal including image data that is obtained by capturing images of the inside of the subject 2 to receiving antennas 51. The capsule endoscope 3 is swallowed into the subject 2, passes through an esophagus of the subject 2, and moves inside the subject 2 due to peristaltic movement of a digestive tract lumen. The capsule endoscope 3 sequentially captures images at a minute time interval, such as 0.5-second time interval (for example, 2 frames per second (fps)) while moving inside the subject 2, generates pieces of image data of the captured images of the inside of the subject 2, and sequentially transmits the pieces of image data to the antenna device 5. The capsule endoscope 3 outputs a radio signal at a certain frequency that is equal to or higher than 300 megahertz (MHz) and equal to or lower than 500 MHz, for example.

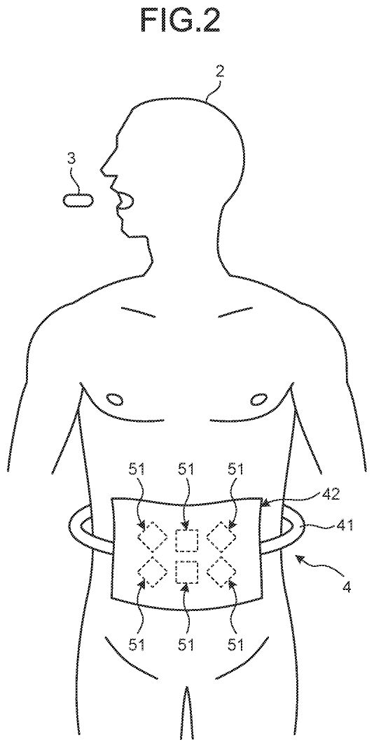

FIG. 2 is a schematic diagram illustrating an overall configuration of the antenna holder illustrated in FIG. 1, and is a diagram illustrating a state in which the antenna holder 4 is attached to the subject 2. As illustrated in FIG. 2, the antenna holder 4 includes a belt unit 41 that fixes the antenna holder 4 to the subject 2, and an antenna mounting unit 42 which is supported by the belt unit 41 and on which the plurality of receiving antennas 51 are mounted.

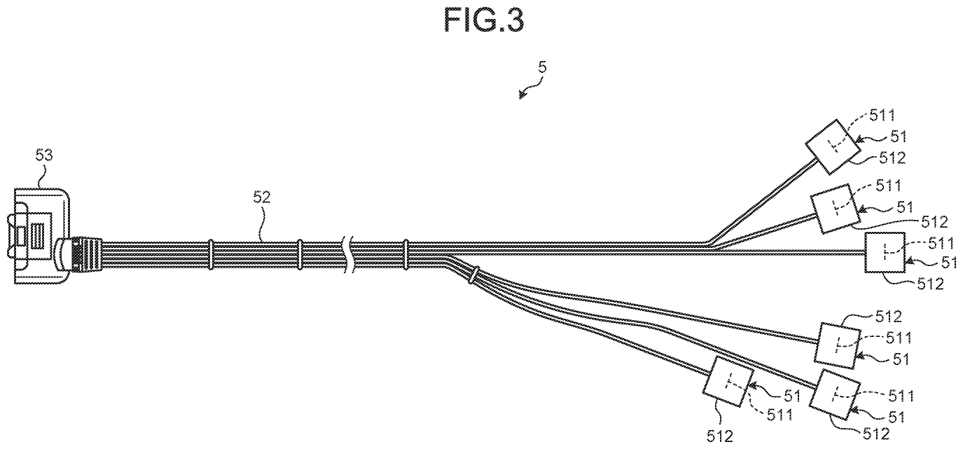

A configuration of the antenna device 5 will be described below with reference to FIG. 3. FIG. 3 is a schematic diagram illustrating an overall configuration of the receiving device illustrated in FIG. 1. As illustrated in FIG. 3, the antenna device 5 includes the plurality of receiving antennas 51 that receive radio signals from the capsule endoscope 3, an antenna cable 52 that transmits the radio signals received by the plurality of receiving antennas 51 to the receiving device 6, and a connector unit 53 that is connected to the receiving device 6. A configuration of the receiving antennas 51 will be described later.

Referring back to FIG. 2, the belt unit 41 of the antenna holder 4 is attached to the subject 2 and fixes the antenna holder 4 along the body surface of the subject 2. It is preferable that the belt unit 41 is made with a stretchable material, such as rubber or polyurethane elastic fiber, so as to be able to fix the antenna holder 4 in accordance with a physical size of the subject.

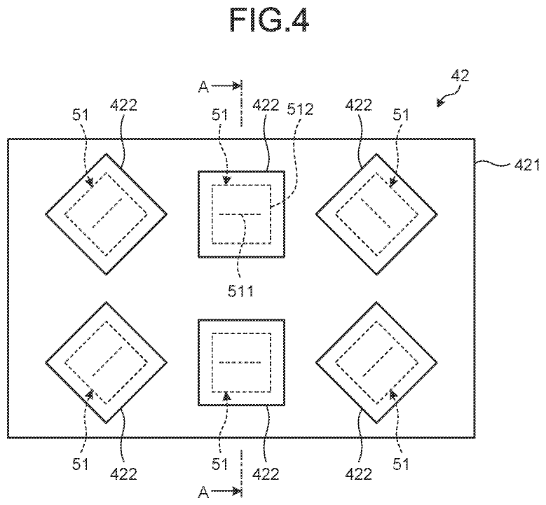

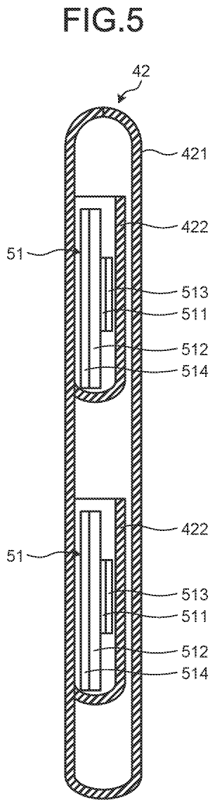

FIG. 4 is an enlarged view of the antenna mounting unit 42 illustrated in FIG. 2. FIG. 5 is a partial cross-sectional view of the antenna mounting unit 42 taken along a line A-A in FIG. 4. In FIG. 5, the subject 2 is located on the left side of the antenna mounting unit 42. Hereinafter explanation will be given based on the assumption that the subject 2 is located on the left side of the subject 2 in other partial cross-sectional views. As illustrated in FIGS. 4 and 5, the antenna mounting unit 42 includes a main body 421 for housing the plurality of receiving antennas 53 and six antenna fixing portions 422 that are arranged inside the main body 421, hold the respective receiving antennas 51, and fix positions of the respective receiving antennas 51. In the following, explanation will be given based on the assumption that the six antenna fixing portions 422 are arranged and the six receiving antennas 51 are mounted, but the number of the antenna fixing portions 422 and the number of the receiving antennas 51 are not limited to six. Further, the number of the antenna fixing portions 422 and the number of the receiving antennas 51 need not always be the same.

The main body 421 is made of fabric or the like and has a sac-like shape. The main body 421 has an openable and closable opening that is made with a point fastener, a line fastener, or a surface fastener, for example.

Each of the antenna fixing portions 422 is made with a cloth, has a pocket-like shape, and contains one of the receiving antennas 51. Each of the antenna fixing portions 422 is arranged in accordance with a position at which each of the receiving antennas 51 is arranged.

Each of the receiving antennas 51 is configured as a substrate, and includes an antenna element 511 that receives a radio signal from the capsule endoscope 3, a first base material 512 on which a wiring pattern for outputting the radio signal received by the antenna element 511 to the receiving device 6 is formed, a second base material 513 that covers the antenna element 511, and a dielectric body 514 that is arranged on one side of the first base material 512 opposite to the antenna element 511. The substrate may be constructed using a flexible substrate having bendability or a rigid substrate having rigidity. The antenna element 511 is configured to include an antenna, such as a dipole antenna.

Each of the first base material 512 and the second base material 513 is made with a material with lower effective permittivity εᵣ than effective permittivity εᵣ of the dielectric body 514. The effective permittivity εᵣ of each of the first base material 512 and the second base material 513 is, for example, equal to or higher than three and equal to or lower than five. Examples of the material of the first base material 512 and the second base material 513 include polyimide and Flame Retardant Type 4 (FR4). In the present embodiment, the antenna element 511 is formed on the first base material 512, and the second base material 513 covers the antenna element 511.

The effective permittivity εᵣ of the dielectric body 514 is higher than the effective permittivity εᵣ of each of the first base material 512 and the second base material 513, and is set to 10 or higher, for example. The dielectric body 514 is constructed using a base material with a board thickness of 10 millimeters (mm) or smaller from the standpoint of easiness of mounting on the receiving antenna 51, for example.

The dielectric body 514 has a certain size capable of covering one surface of the first base material 512. In this case, it is sufficient that the dielectric body 514 has a size equal to or larger than a principal surface of the first base material 512. Here, the “principal surface” is a surface with the largest area among six surfaces of the substrate. Further, the dielectric body 514 has a certain size such that when an outer edge of the dielectric body 514 is projected on the first base material 512, the first base material 512 is included in a projected region. The dielectric body 514 is located on the subject 2 side in the receiving antenna 51 when the antenna holder 4 is attached to the subject 2. In other words, the dielectric body 514 covers a surface at the side of the subject 2 in the receiving antenna 51.

Meanwhile, in the present embodiment, explanation will be given based on the assumption that the dielectric body 514 is fixed to the first base material 512, but the dielectric body 514 may be arranged in an attachable/detachable manner. The dielectric body 514 may be fixed by using a well-known fixing method, such as adhesive or screwing.

Further, in the receiving antenna 51, a size of the antenna element 511 (an area of a principal surface) is smaller than a size of the first base material 512 and the size of the dielectric body 514 and equal to the size of the second base material 513.

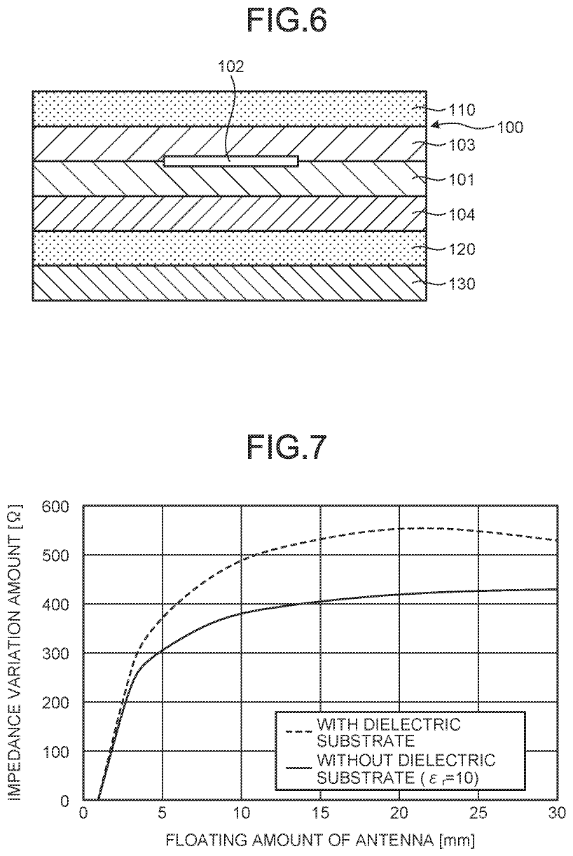

FIG. 6 is a diagram for explaining a specimen for analyzing a difference in impedance variation. A specimen 100 illustrated in FIG. 6 includes a first base material 101, an antenna element 102 formed on the first base material 101, a second base material 103 arranged on a surface of the first base material 101 on which the antenna element 102 is mounted, and a dielectric body 104 arranged on another surface of the first base material 101 on the opposite side of the surface on which the antenna element 102 is mounted. A dipole antenna is mounted on the antenna element 102. The antenna element 102 is sandwiched between the first base material 101 and the second base material 103. The dielectric body 104 has a board thickness of 1 mm. The first base material 101 and the second base material 103 respectively correspond to the first base material 512 and the second base material 513 as described above, and the dielectric body 104 corresponds to the dielectric body 514.

Further, as a comparative example of the specimen that includes the dielectric body 104, a specimen that does not include the dielectric body 104, that is, a specimen formed of the first base material 101, the antenna element 102, and the second base material 103 was also prepared.

With use of the specimens as described above, a floating amount of the antenna was changed from 1 mm to 30 mm, and a variation amount of characteristic impedance (impedance variation amount (Ω)) was analyzed with respect to each of the floating amount. The analysis was performed using a simulation tool (Advanced Design System (ADS) 2016). In the analysis, a finite air layer 110 was set on one side of the second base material 103 opposite to the first base material 101 (the antenna element 102). Further, a finite air layer 120 and a phantom layer 130 were set on one side of the dielectric body 104 opposite to the first base material 101. The phantom layer 130 corresponds to a subject. Meanwhile, the floating amount of the antenna indicates a distance between a device that generates a radio signal and the antenna, and in this case, corresponds to a distance between a surface of the first base material 101 (the first base material 512) at the side of the dielectric body 10-1 (the dielectric body 514) and an outer surface of the phantom layer 130 (subject). Furthermore, the first base material 101 and the second base material 103 were made with polyimide.

FIG. 7 is a graph for explaining a difference in impedance variation between with and without the dielectric body. FIG. 7 illustrates a case in which the effective permittivity εᵣ of the dielectric body 104 is set to 10. As illustrated in FIG. 7, in both of the two specimens, the impedance variation amounts increase with increase in the floating amount of the antenna. However, the variation amount of the specimen 100 that includes the dielectric body 104 is smaller than the variation amount of the specimen that does not include the dielectric body. A variation amount of receiving power that is received by the antenna decreases with decrease in the variation amount of the characteristic impedance, and therefore, it may be said that the specimen 100 including the dielectric body 104 is able to perform communication more stably.

Moreover, the variation amount of the characteristic impedance decreases with increase in the effective permittivity of the dielectric body 104.

Referring back to FIG. 1, the receiving device 6 records image data of the inside of the subject, where the image data is included in the radio signal that is transmitted from the capsule endoscope 3 via the plurality of receiving antennas 51, or the receiving device 6 displays an image corresponding to the image data of the inside of the subject 2. The receiving device 6 includes a receiving display unit 61 that displays an image corresponding to the image data, and an operating unit 62 that receives an instruction to operate the receiving device and receives input of information on the position of each of the receiving antennas 51. Further, the receiving device 6 receives a radio signal that is transmitted from the capsule endoscope 3 via each of the receiving antennas 51, calculates and records reception intensity (reception electric field intensity) of the received radio signal for each of the receiving antennas 51, and estimates a position of the capsule endoscope 3 inside the subject 2. The receiving device 6 records image data included in the radio signal received from the capsule endoscope 3, the reception intensity of the radio signal received by each of the receiving antennas 51, and time information on image data generated by the capsule endoscope 3, in an associated manner.

The image processing apparatus 7 displays an image corresponding to the image data of the inside of the subject 2, where the image data is obtained via the receiving device 6. The image processing apparatus 7 includes a cradle 71 that reads image data or the like from the receiving device 6, an operation input unit 72, such as a mouse 72a and a keyboard 72b, and a display unit 73 that displays an image corresponding to the image data. When the receiving device 6 is attached to the cradle 71, the cradle 71 acquires image data and acquires the reception intensity of each of the receiving antennas 51, the time information on the image data generated by the capsule endoscope 3, identification information on the capsule endoscope 3, and the like, which are associated with the image data, from the receiving device 6, and then transfers various kinds of the acquired information to the image processing apparatus 7. The operation input unit 72 receives input from a user. The user operates the operation input unit 72, observes living body sites, such as an esophagus, a stomach, a small intestine, and a large intestine, inside the subject 2, and makes a diagnosis on the subject 2 while viewing images of the inside of the subject 2 that are sequentially displayed by the image processing apparatus 7.

In the embodiment as described above, the dielectric body 514 having higher effective permittivity than the effective permittivity of each of the first base material 512 and the second base material 513 included in the antenna element 511 is arranged in each of the receiving antennas 51. Therefore, even if the receiving antenna 51 floats from the subject 2, and a distance between the receiving antenna 51 and the subject 2 increases, it is possible to suppress variation in characteristic impedance as compared to a case in which the dielectric body is not provided. According to the present embodiment, it is possible to stably perform radio communication even if the distance between the subject and the receiving antenna is changed.

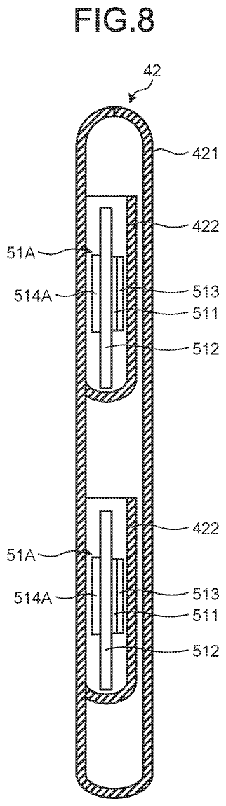

FIG. 8 is a partial cross-sectional view schematically illustrating a configuration of a main part of an antenna holder that holds receiving antennas according to a first modification of the embodiment. The partial cross-sectional view illustrated in FIG. 8 corresponds to a cross section taken along a line A-A illustrated in FIG. 4. In the embodiment as described above, it is explained that the dielectric body 514 covers the entire first base material 512; however, in the first modification, a dielectric body 514A covers a part of the first base material 512. A receiving antenna 51A according to the first modification includes the antenna element 511, the first base material 512, the second base material 513, and the dielectric body 514A that is arranged on one side of the first base material 512 opposite to the antenna element 511. In the following, a configuration (the dielectric body 514A) different from the embodiment as described above will be described.

Similarly to the dielectric body 514 as described above, effective permittivity εᵣ of the dielectric body 514 is higher than the effective permittivity εᵣ of each of the first base material 512 and the second base material 513, and is set to 10 or higher.

The dielectric body 514A has a size capable of covering a part of one surface of the first base material 512. Further, the dielectric body 514A is located on the subject 2 side in the receiving antenna 51A when the antenna holder 4 is attached to the subject 2. In other words, the dielectric body 514A covers a part of a surface at the side of the subject 2 in the receiving antenna 51A.

Even in the first modification, the dielectric body 514A intervenes communication between the antenna element 511 and the capsule endoscope 3, so that even if a distance between the subject and the receiving antenna is changed, it is possible to stably perform radio communication.

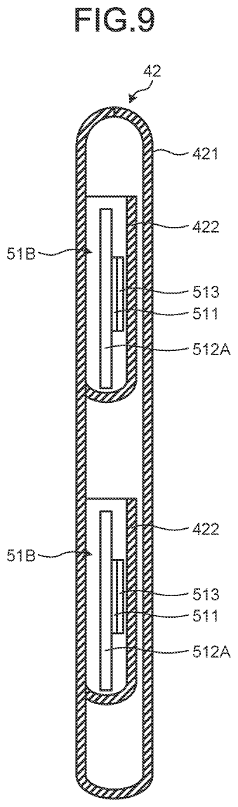

FIG. 9 is a partial cross-sectional view schematically illustrating a configuration of a main part of an antenna holder that holds receiving antennas according to a second modification of the embodiment. The partial cross-sectional view illustrated in FIG. 9 corresponds to a cross section taken along a line A-A illustrated in FIG. 4. In the embodiment as described above, an example has been described in which the dielectric body 514 having relatively high effective permittivity is arranged in the receiving antenna 51; however, in the second modification, a first base material 512A itself is made with a material having high effective permittivity. A receiving antenna 51B according to the second modification includes the antenna element 511, the first base material 512A, and the second base material 513. In the following, a configuration (the first base material 512A) different from the embodiment as described above will be described.

The first base material 512A is made with a material having certain effective permittivity εᵣ that is higher than the effective permittivity εᵣ of the second base material 513 and that is set to, for example, 10 or higher, similarly to the dielectric body 514 as described above.

Even in the second modification, the first base material 512A having high effective permittivity intervenes communication between the antenna element 511 and the capsule endoscope 3, so that even if a distance between the subject and the receiving antenna is changed, it is possible to stably perform radio communication. Further, according to the second modification, because the dielectric body is not arranged, it is possible to reduce a size as compared to the embodiment as described above.

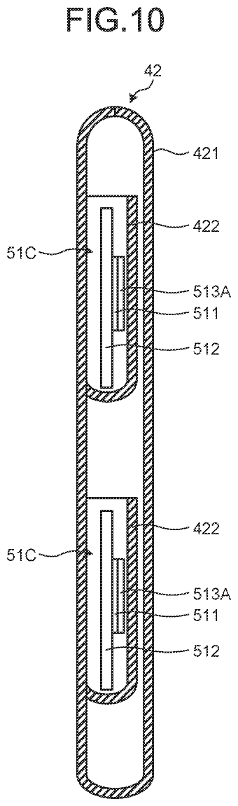

FIG. 10 is a partial cross-sectional view schematically illustrating a configuration of a main part of an antenna holder that holds receiving antennas according to a third modification of the embodiment. The partial cross-sectional view illustrated in FIG. 10 corresponds to a cross section taken along a line A-A illustrated in FIG. 4. In the embodiment as described above, an example has been described in which the dielectric body 514 having relatively high effective permittivity is arranged in the receiving antenna 51; however, in the third modification, a second base material 513A itself is made with a material having high effective permittivity. A receiving antenna 51C according to the third modification includes the antenna element 511, the first base material 512, and the second base material 513A. In the following, a configuration (the second base material 513A) different from the embodiment as described above will be described.

The second base material 513A is made with a material having certain effective permittivity εᵣ that is higher than the effective permittivity εᵣ of the first base material 512 and that is set to, for example, 10 or higher, similarly to the dielectric body 514 as described above.

Even in the third modification, the receiving antenna 51C includes the second base material 513A having high effective permittivity for communication between the antenna element 511 and the capsule endoscope 3, so that even if a distance between the subject and the receiving antenna is changed, it is possible to stably perform radio communication. Further, according to the third modification, because the dielectric body is not arranged, it is possible to reduce a size as compared to the embodiment as described above.

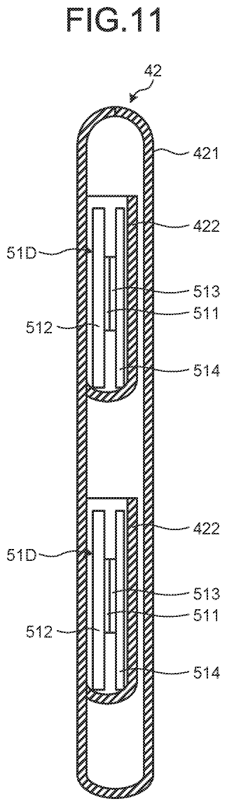

FIG. 11 is a partial cross-sectional view schematically illustrating a configuration of a main part of an antenna holder that holds receiving antennas according to a fourth modification of the embodiment. The partial cross-sectional view illustrated in FIG. 11 corresponds to a cross section taken along a line A-A illustrated in FIG. 4. In the embodiment as described above, an example has been described in which the dielectric body 514 is mounted on the first base material 512 in the receiving antenna 51; however, in the fourth modification, the dielectric body 514 is mounted on the second base material 513. A receiving antenna 51D according to the fourth modification includes the antenna element 511, the first base material 512, the second base material 513, and the dielectric body 514. Each of the components is the same as that of the embodiment as described above, and arrangement of the components will be described below.

The dielectric body 514 is arranged on one side of the second base material 513 opposite to the antenna element 511. Further, the dielectric body 514 is located on an opposite side of the subject 2 in the receiving antenna 51D when the antenna holder 4 is attached to the subject 2.

Even in the fourth modification, the receiving antenna 51D includes the dielectric body 514 having high effective permittivity for communication between the antenna element 511 and the capsule endoscope 3, so that even if a distance between the subject and the receiving antenna is changed, it is possible to stably perform radio communication.

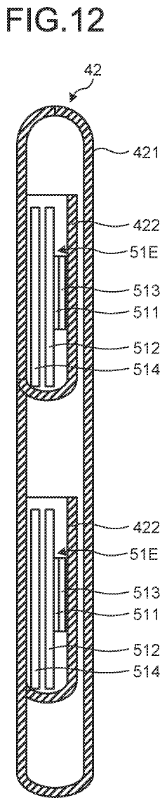

FIG. 12 is a partial cross-sectional view schematically illustrating a configuration of a main part of an antenna holder that holds receiving antennas according to a fifth modification of the embodiment. The partial cross-sectional view illustrated in FIG. 12 corresponds to a cross section taken along a line A-A illustrated in FIG. 4. In the embodiment as described above, an example has been described in which the dielectric body 514 is mounted on the first base material 512 in the receiving antenna 51; however, in the fifth modification, the dielectric body 514 that is separated from the first base material 512, the antenna element 511, and the second base material 513 is housed in each of the antenna fixing portions 422.

In the fifth modification, a receiving antenna 51E that includes the antenna element 511, the first base material 512, and the second base material 513 is provided, and the dielectric body 514 that is separated from the receiving antenna 51E is also provided. The receiving antenna 51E and the dielectric body 514 are housed in each of the antenna fixing portions 422. Each of the components is the same as that of the embodiment as described above, and arrangement of the components will be described below.

The antenna element 511 and the second base material 513 are provided on one side of the first base material 512, while the dielectric body 514 is arranged opposite to the one side of the first base material 512. Further, the dielectric body 514 is located on the subject 2 side in the receiving antenna 51E when the antenna holder 4 is attached to the subject 2.

Even in the fifth modification, the dielectric body 514 having high effective permittivity is arranged in the vicinity of the receiving antenna 51E for communication between the antenna element 511 and the capsule endoscope 3, so that even if a distance between the subject and the receiving antenna is changed, it is possible to stably perform radio communication.

Meanwhile, while it is explained, in the embodiment as described above, that the dielectric body 514 is mounted on the first base material 512, it may be possible to combine, for example, the embodiment and the fourth modification such that the antenna element 511, the first base material 512, and the second base material 513 are sandwiched between the two dielectric bodies 514.

Furthermore, while the capsule endoscope 3 is described as one example of the medical device in the embodiment as described above, the medical device is not limited to this example. For example, it may be possible to adopt a device that is introduced into a subject, acquires pH information, and outputs the information as a radio signal.

As described above, the receiving antenna system and the capsule endoscope system according to the present disclosure are useful for stably performing radio communication even if a distance between the subject and the receiving antenna is changed.

According to the present disclosure, it is possible to stably perform radio communication even if a distance between a subject and a receiving antenna is changed.

Additional advantages and modifications will readily occur to those skilled in the art. Therefore, the disclosure in its broader aspects is not limited to the specific details and representative embodiments shown and described herein. Accordingly, various modifications may be made without departing from the spirit or scope of the general inventive concept as defined by the appended claims and their equivalents.

What is claimed is:

1. A receiving antenna system comprising:
a receiving antenna including:
an antenna element configured to receive a radio signal transmitted from a medical device introduced into a subject; and
a base material on which the antenna element is mounted/the base material including
a first base material, and
a second base material; and
a holder attached to the subject and configured to house the receiving antenna,
wherein the antenna element is mounted on the first base material or the second base material,
one of the first base material and the second base material has higher effective permittivity than effective permittivity of another one of the first base material and the second base material, and
the first base material, the antenna element and the second base material are arranged in this order from a subject side.

2. The receiving antenna system according to claim 1, further comprising a dielectric body having higher effective permittivity than effective permittivity of the first base material.

3. The receiving antenna system according to claim 1, wherein the first base material has higher effective permittivity than effective permittivity of the second base material.

4. The receiving antenna system according to claim 1, wherein the second base material has higher effective permittivity than effective permittivity of the first base material.

5. The receiving antenna system according to claim 2, wherein
the dielectric body has higher effective permittivity than the effective permittivity of the first base material and effective permittivity of the second base material, and
the dielectric body, the first base material, the antenna element, and the second base material are arranged in this order from the subject side when the receiving antenna is housed in the holder and the holder is attached to the subject.

6. The receiving antenna system according to claim 2, wherein
the dielectric body has higher effective permittivity than effective permittivity of the second base material, and
the first base material, the antenna element, the second base material, and the dielectric body in this order from the subject side when the receiving antenna is housed in the holder and the holder is attached to the subject.

7. The receiving antenna system according to claim 1, wherein the effective permittivity of the part of the receiving antenna is set to 10 or higher.

8. The receiving antenna system according to claim 1, wherein the antenna element receives the radio signal at a certain frequency that is equal to or higher than 300 MHz and equal to or lower than 500 MHz.

9. The receiving antenna system according to claim 2, wherein the dielectric body has a board thickness of 10 millimeters or smaller.

10. A receiving antenna system comprising:
a receiving antenna including:
an antenna element configured to receive a radio signal transmitted from a medical device introduced into a subject; and
a base material on which the antenna element is mounted, the base material including
a first base material, and
a second base material; and
a dielectric body having higher effective permittivity than effective permittivity of the base material; and
a holder attached to the subject and configured to house the receiving antenna and the dielectric body,
wherein the antenna element is mounted on the first base material or the second base material, and
the dielectric body, the first base material, the antenna element and the second base material are arranged in this order from a subject side.

11. A capsule endoscope system comprising:
a capsule endoscope that is introduced into a subject and transmits a radio signal;
a receiving antenna including:
an antenna element configured to receive the radio signal; and
a base material on which the antenna element is mounted, the base material including
a first base material, and
a second base material; and
a holder attached to the subject and configured to house the receiving antenna,
wherein the antenna element is mounted on the first base material or the second base material,
one of the first base material and the second base material has higher effective permittivity than effective permittivity of another one of the first base material and the second base material, and
the first base material, the antenna element and the second base material are arranged in this order from a subject side.
